(19) United States
(12) Patent Application Publication
(10) Pub. No.: US 2010/0196402 A1
(43) Pub. Date: Aug. 5, 2010

(54) Method for Predicting the Response of a Patient to Treatment with an Anti-TNF Alpha Antibody

(75) Inventors: Michael Ehrenstein, London (GB); Claudia Mauri, London (GB)

Correspondence Address: CLARK & ELBING LLP, 101 FEDERAL STREET, BOSTON, MA, 02110, US

(73) Assignee: UCL BUSINES PLC, LONDON (GB)

(21) Appl. No.: 12/601,173

(22) PCT Filed: May 20, 2008
(86) PCT No.: PCT/GB08/01728

§ 371 Date: Apr. 12, 2010

(30) Foreign Application Priority Data
May 22, 2007 (GB) 0709803.1

(51) Int. Cl. A61K 39/395; C12Q 1/02; A61P 29/00; A61P 37/00

(52) U.S. Cl. 424/173.1; 435/29

(57) ABSTRACT

The present invention relates to a method for predicting the response of a patient to treatment with an anti-TNFα therapy, in particular an anti-TNFα antibody, the method comprising: (a) providing an in vitro sample of T cells from the patient; (b) exposing said T cells to an anti-TNFα therapy; and (c) determining whether regulatory T cells are induced in said sample of T cells wherein the induction of regulatory T cells indicates that the patient is likely to respond to treatment with said anti-TNFα therapy.

### FIELD OF THE INVENTION

The present invention relates to methods to allow the selection of patients that will respond to particular anti-TNFα therapies. These methods allow the use of such therapies only for those patients that will respond to them, and avoid the unnecessary cost and potential side-effects that might be incurred in patients that will not respond to such a therapy.

### BACKGROUND TO THE INVENTION

Autoimmunity is the failure of an organism to recognize its own constituent parts, which results in an immune response against the organism's own cells and tissues. Any disease that results from such an aberrant immune response is termed an autoimmune disease.

Current treatments for autoimmune disease are largely palliative, generally immunosuppressive, or anti-inflammatory. Specific immunomodulatory therapies, such as the TNFα antagonist etanercept and the antibody-based drugs infliximab and adalimumab, have been shown to be useful in treating autoimmune diseases such as rheumatoid arthritis.

Progress in the understanding of pro-inflammatory mediators important in the pathogenesis of rheumatoid arthritis has heralded a new era of targeted therapy. The first and most successful example is that of infliximab, which binds to TNFα and demonstrates substantial therapeutic efficacy by decreasing inflammation and slowing joint destruction in 60-70% of patients with rheumatoid arthritis (Maini, R., et al. 1999 Lancet. 354:1932-1939; Maini, R. N., et al. 2004 Arthritis Rheum. 50:1051-1065).

### SUMMARY OF THE INVENTION

The inventors have found that “natural” regulatory T cells (Treg) isolated from patients with active rheumatoid arthritis display a reduced suppressive capacity. After anti-TNFα treatment, the Treg pool appear to regain their suppressive function. However, rather than restoring a defect in these Treg, the anti-TNFα treatment has been found to induce the differentiation (in vivo and in vitro) of a distinct and potent population of Treg from responder T cells (CD4⁺CD25⁻) via TGFβ. These findings indicate that the therapeutic effect of anti-TNFα agents such as infliximab may be at least partly based on the induction of Tregs, and suggest that anti-TNFα therapies such as infliximab have the potential to restore tolerance in patients with rheumatoid arthritis.

The differentiation of Tregs can therefore be used as a biomarker to predict the response of a patient to therapy in diseases such as rheumatoid arthritis.

Accordingly, the present invention provides a method for predicting the response of a patient to treatment with an anti-TNFα antibody, the method comprising:
(a) providing an in vitro sample of T cells from the patient;
(b) exposing said T cells to an anti-TNFα antibody; and
(c) determining whether regulatory T cells are induced in said sample of T cells wherein the induction of regulatory T cells indicates that the patient is likely to respond to treatment with said anti-TNFα antibody.

These methods can be used to determine which patients should be treated with a given anti-TNFα antibody. Thus, the invention also provides a method for treating an inflammatory or autoimmune disease, the method comprising:
(a) using a method of the invention to identify a patient as being likely to respond to treatment with an anti-TNFα antibody; and
(b) treating the patient with said anti-TNFα antibody.

The invention also provides an anti-TNFα antibody for use in the treatment of an inflammatory or autoimmune disease in a patient, wherein said patient has been identified as being likely to respond to treatment with an anti-TNFα antibody using a method of the invention.

### BRIEF DESCRIPTION OF THE DRAWING

FIG. 1 shows the percentage of regulatory T cells (CD4⁺Foxp3⁺) in PBMC from healthy controls, patients with active RA and RA patients treated with anti-TNFα therapies. Only responding patients are shown for each of the three anti-TNFα treated groups.

### DETAILED DESCRIPTION OF THE INVENTION

The present invention derives from the inventors' finding that it is possible to predict, based on the results of a preliminary test, whether a patient is likely to respond to a particular therapeutic regime. In particular, the invention relates to the prediction of whether a patient will respond to an anti-TNFα therapy.

A number of such anti-TNFα therapies are known. However, these therapies are not effective in all patients. For example, infliximab, which is discussed further below, is used in the therapy of a number of inflammatory and autoimmune diseases, but not all patients respond to treatment with infliximab. For example, in the treatment of rheumatoid arthritis, infliximab shows substantial therapeutic efficacy by decreasing inflammation and slowing joint destruction in 60-70% of patients. In the remaining 30-40% of patients, however, this therapeutic effect is not seen. It would clearly be beneficial to identify whether a particular patient will fall in the 60-70% of individuals where such a therapy will be effective, or the 30-40% of individuals where such a therapy will be ineffective.

Such a predictive test would be of financial benefit since it would be unnecessary to use potentially expensive anti-TNF drugs on patients that will not respond to them. This can therefore significantly increase the success rate of such a drug because the use of the drug can be targeted to susceptible patients. Such a predictive test can also be used to help decide on the treatment of patients at an early stage of disease. For example, there is evidence that use of infliximab in patients with early-stage rheumatoid arthritis can lead to remission. Because of the improved success rate that can be achieved by using a predictive test of the invention prior to a decision on treatment, the use of anti-TNFα therapies on such early stage patients becomes a more viable and more justifiable option and can be expected to lead to a significant clinical benefit. A predictive test would also be of particular benefit to patients that do not respond to the anti-TNFα therapy, since many such anti-TNFα therapies can be associated with side effects. By using a predictive test of the invention, it can be ensured that patients who will not be responsive to such a therapy are not exposed to such risks unnecessarily.

The present invention therefore describes a predictive test, which can be used to determine whether a patient is likely to respond to treatment with a particular anti-TNFα therapy. A method for predicting the response of a patient to treatment with an anti-TNFα therapy may thus comprise: (a) providing a sample of T cells from the patient; (b) exposing said T cells to an anti-TNFα therapy; and (c) determining whether regulatory T cells are induced in said sample of T cells; wherein the induction of regulatory T cells indicates that the patient is likely to respond to treatment with said anti-TNFα therapy.

In particular, the present invention provides a method for predicting the response of a patient to treatment with an anti-TNFα antibody, the method comprising: (a) providing an in vitro sample of T cells from the patient; (b) exposing said T cells to an anti-TNFα antibody; and (c) determining whether regulatory T cells are induced in said sample of T cells, wherein the induction of regulatory T cells indicates that the patient is likely to respond to treatment with said anti-TNFα antibody.

Anti-TNFα Therapies

Tumor necrosis factor-alpha (TNFα) is a cytokine produced by monocytes and macrophages. It mediates the immune response by increasing the transport of white blood cells to sites of inflammation, and through additional molecular mechanisms which initiate and amplify inflammation. Biological activities that are attributed to TNFα include: induction of proinflammatory cytokines such as IL-1 and IL-6, enhancement of leukocyte movement or migration from the blood vessels into the tissues by increasing the permeability of endothelial layer of blood vessels; and increasing the release of adhesion molecules.

Because of its key role in the regulation of inflammation, TNFα is a key target for a number of therapies, particularly those directed at preventing or reducing inflammation or autoimmune disorders.

The present invention relates to such therapies that are directed against the TNFα response. In particular, the present invention relates to anti-TNFα therapies. By an anti-TNFα therapy is meant a therapy or treatment that is directed against TNFα. For example, an anti-TNFα therapy may prevent or reduce the production or release of TNFα. An anti-TNFα therapy may prevent or reduce the activity of TNFα. An anti-TNFα therapy may involve the use of a molecule that specifically binds to TNFα. Such a molecule may prevent the activity of TNFα. An anti-TNFα therapy may be capable of neutralising or removing extracellular TNFα and/or transmembrane TNFα and/or receptor bound TNFα. An anti-TNFα therapy may utilise a molecule that binds to soluble TNFα (e.g. free floating in the blood) and/or transmembrane TNFα (that may be present at the surface of T cells and other immune cells). An anti-TNFα therapy may utilise a molecule that inhibits or prevents effective binding of TNFα with its receptors. For example, in one embodiment the anti-TNFα therapy neutralises, binds to or removes membrane bound TNFα.

An anti-TNFα therapy is preferably specific to TNFα, that is it preferably acts exclusively on TNFα, or acts on TNFα in preference to other molecules. For example, an anti-TNFα therapy preferably acts on TNFα, but not TNFβ, even though the two types of TNF can utilise the same receptors.

In accordance with the present invention, the anti-TNFα therapy preferably utilizes a molecule that is capable of binding to, and neutralizing, TNFα. Preferred molecules are capable of neutralizing all forms of TNFα, for example extracellular TNFα, transmembrane TNFα and receptor-bound TNFα. In particular, preferred anti-TNF therapies for use in accordance with the present invention are capable of neutralizing receptor-bound TNFα. Anti-TNFα therapies for use in accordance with the present invention preferably have the capacity of lysing cells involved in the inflammatory process.

Accordingly, a preferred group of anti-TNFα therapies for use in accordance with this invention are anti-TNFα antibodies. Such an antibody may be monoclonal or polyclonal or may be an antigen-binding fragment thereof. For example, an antigen-binding fragment may be or comprise a F(ab)₂, Fab or Fv fragment, i.e. a fragment of the “variable” region of the antibody, which comprises the antigen binding site. An antibody or fragment thereof may be a single chain antibody, a chimeric antibody, a CDR grafted antibody or a humanised antibody.

An antibody may be directed to the TNFα molecule, i.e. it may bind to epitopes present on TNFα and thus bind selectively and/or specifically to TNFα. An antibody may be directed to another molecule that is involved in the expression and/or activity of TNFα. For example, a polyclonal antibody may be produced which has a broad spectrum effect against one or more epitopes on TNFα and/or one or more other molecules that are involved in the expression and/or activity of TNFα.

Antibodies can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Infliximab and adalimumab are examples of antibodies capable of neutralizing all forms (extracellular, transmembrane, and receptor-bound) of TNFα. Accordingly, in a preferred aspect of the invention, the anti-TNFα therapy comprises, consists essentially or or consists of the administration of infliximab and/or adalimumab. Etanercept, another TNFα antagonist, is a different type of molecule (receptor-construct fusion protein), and because of its modified form, cannot neutralize receptor-bound TNFα. Anti-TNFα antibodies such as infliximab and adalimumab also have the capability of lysing cells involved in the inflammatory process, whereas the receptor fusion protein etanercept apparently lacks this capability.

Infliximab (sold under the brand name Remicade®) is a drug used to treat inflammatory and autoimmune disorders. Infliximab is a chimeric monoclonal antibody comprising murine binding VK and VH domains and human constant Fc domains.

Infliximab neutralizes the biological activity of TNFα by binding with high affinity to the soluble (free floating in the blood) and transmembrane (located on the outer membranes of T cells and similar immune cells) forms of TNFα and inhibits or prevents the effective binding of TNFα with its receptors. Infliximab has high specificity for TNFα, and does not neutralize TNFβ, although TNFβ utilizes the same receptors as TNFα.

To date, infliximab has been approved by the U.S. Food and Drug Administration for the treatment of psoriasis, pediatric Crohn's disease, ankylosing spondylitis, Crohn's disease, psoriatic arthritis, rheumatoid arthritis, and ulcerative colitis.

Adalimumab (sold under the brand name Humira®) also binds to TNFα, preventing it from activating TNF receptors. Adalimumab was constructed from a fully human monoclonal antibody, while infliximab is a mouse-human chimeric antibody. To date, adalimumab has been approved by the United States Food and Drug Administration (FDA) for the treatment of rheumatoid arthritis, psoriatic arthritis, alkylosing sponylitis and Crohn's disease, and an application has been made to the FDA for approval in the treatment of plaque psoriasis.

Etanercept is a recombinant human soluble TNFα receptor fusion protein. It is a large molecule, with a molecular weight of 150 kDa., that binds to TNFα and decreases its role in disorders involving excess inflammation in humans and other animals, including autoimmune diseases such as ankylosing spondylitis, juvenile rheumatoid arthritis, psoriasis, psoriatic arthritis, rheumatoid arthritis, and, potentially, in a variety of other disorders mediated by excess TNFα.

There are two types of TNF receptors: those found embedded in white blood cells that respond to TNF by releasing other cytokines, and soluble TNF receptors which are used to deactivate TNF and blunt the immune response. In addition, TNF receptors are found on the surface of virtually all nucleated cells (red blood cells, which are not nucleated, do not contain TNF receptors on their surface). Etanercept mimics the inhibitory effects of naturally occurring soluble TNF receptors, the difference being that etanercept, because it is a fusion protein rather than a simple TNF receptor, has a greatly extended half-life in the bloodstream, and therefore a more profound and long-lasting biologic effect than than a naturally occurring soluble TNF receptor.

Etanercept is made from the combination of two naturally occurring soluble human 75-kilodalton TNF receptors linked to an Fc portion of an IgG1. The effect is an artificially engineered dimeric fusion protein. As shown in the Examples, treatment of a sample with etanercept did not lead to an alternation in Treg numbers despite a clinical response in patients to that therapy. The anti-TNFα therapy used as described herein is therefore not intended to include treatment with etanercept alone, although etanercept therapy may be used in combination with another anti-TNFα therapy, such as an anti-TNFα antibody. In one embodiment, the anti-TNFα therapy is not a molecule that mimics a naturally occurring TNF receptor.

### Patients to be Treated

The present invention relates in particular to the treatment or prevention of diseases or other conditions which are associated with the presence of TNFα. For example, the invention relates to the treatment or prevention of inflammatory or autoimmune diseases. The patient to be treated is typically suffering from such an inflammatory or autoimmune disease. The patient is typically an individual for whom treatment using an anti-TNFα therapy is being considered. For example, anti-TNFα therapies are currently used in the treatment of rheumatoid arthritis, psoriatic arthritis, Crohn's disease, psoriasis, ankylosing spondylitis and ulcerative colitis. The methods of the present invention may be used to assist the treatment of a patient with any one of these diseases, or any other disease, condition or symptom that is treatable using an anti-TNFα therapy.

These treatments may be used on any animal which is susceptible to such a disease. The subject to be treated may be any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. In a preferred embodiment, the subject is a human. The terms do not denote a particular age. Thus, for example, adult, juvenile and newborn subjects are all intended to be covered.

The methods described herein are intended for use in any of the above vertebrate species, since the immune systems of all of these vertebrates operate similarly. If a mammal, the subject will preferably be a human, but may also be a domestic livestock animal, laboratory subject or pet animal. The methods described herein may thus be used in the diagnosis or treatment of any such species.

A suitable therapy may be selected based upon the species to be treated. For example an anti-TNFα antibody for use in treating a particular species may be modified to be active or more effective in that species. An anti-TNFα antibody for use in the treatment of a human may be a human monoclonal antibody, may be a humanised antibody, may be a chimeric antibody comprising human domains, or may be a CDR grafted antibody.

Sample from a Patient

The present invention relates to methods to be carried out on a sample from the patient of interest. Preferably the method is carried out in vitro on a sample that has been obtained from a patient. As discussed herein, the patient is typically suffering from an inflammatory or autoimmune disease. The patient is typically an individual for whom treatment using an anti-TNFα therapy is being considered. The method of the present invention is carried out on a sample from such a patient. Preferably the sample is obtained from the patient before treatment for the disease or condition is started. For example, the sample may be obtained from a patient before any treatment with an anti-TNFα therapy is begun.

The sample may be from any tissue or bodily fluid that comprises T cells. For example, the sample may be a blood sample or a sample of synovial fluid such as from an inflamed joint.

The sample may be treated before use in a method of the invention either to remove components that might interfere with the method or to make the results of the method easier to assess. For example, a sample may be treated to remove components other than T cells or to select or exclude particular types of cell from the sample. A sample for use in a method of the invention may therefore comprise a purified, substantially purified, isolated or substantially isolated population of T cells.

A sample of T cells for use in a method of the invention may comprise a population of responder T cells. For example, a sample of T cells for use in a method of the invention may consist of or substantially consist of a population of responder T cells. A sample of T cells may be a population that is predominantly responder T cells, such as at least 50%, at least 60%, at least 70%, at least 80%, at least 90%, at least 95% or more responder T cells.

A responder T cell is a cell that can be regulated by a regulatory T cell. A responder T cell proliferates upon stimulation through the T cell receptor and can secrete pro-inflammatory cytokines. For example a responder T cell may be identified as a T cell that secretes IL-2. A responder T cell may be identified as a T cell that lacks the features present in a regulatory T cell. A population of responder T cells may thus be identified by selecting cells within a sample that are CD4⁺ and/or CD25⁻ and/or CD127⁺ and/or Foxp3⁻. One, two, three or all four of these features may be assessed. A population of responder T cells may be obtained from a sample by sorting cells within the sample for cells having particular characteristics. For example, cells may be sorted by fluorescence-activated cell-sorting (FACS) based on the presence or absence of one or more identifying molecules in the cells. A population of responder T cells may be identified by FACS sorting for cells that are CD4⁺ and CD25⁻ and CD127⁺.

According to the present invention, a sample as described herein is exposed to an anti-TNFα therapy, such as an anti-TNFα antibody. The exposure to anti-TNFα therapy may be carried out using any suitable set of conditions, based on the particular therapy being tested. Preferably this exposure is carried out in vitro by contacting the sample with an anti-TNFα agent. The sample may be treated prior to or during such exposure to enhance any response to the therapy. For example, a population comprising responder T cells may be treated to activate the cells. For example, responder T cells may be activated over a period of time, such as overnight, with anti-CD3/CD28 in the presence or absence of an anti-TNFα therapeutic agent.

A method of the invention may also comprise a control in which a further sample is treated in the same way as the experimental sample, but in the absence of the anti-TNFα therapy, such as in the absence of an anti-TNFα therapeutic agent. A suitable control may be readily identified depending on the particular anti-TNFα therapy being used. This may involve treatment with the diluent(s) used for formulation of an anti-TNFα therapy. For example, where the anti-TNFα therapy is an anti-TNFα antibody, two identical samples may be used, one treated with the antibody of interest, and the other treated in the same way, but in the absence of the antibody. Such a control will generally be treated in parallel with the experimental sample and may be used to ensure that any induction in Treg caused by the therapy is actually the result of the anti-TNFα therapeutic agent, and not caused by other conditions or substances involved in the treatment.

The dose of anti-TNFα therapy to be used in accordance with the invention will depend upon the nature of the specific therapy. A suitable dose can be determined by a skilled practitioner based on his common general knowledge, taking into account, for example, the regime and dose that would be used for in vivo treatment using that therapy. For example, a suitable dose may be selected to reflect the level of a therapeutic agent that would be present in the blood circulatory system of a patient after in vivo administration.

### Regulatory T Cells

According to the present invention, a sample as described herein is exposed to an anti-TNFα therapy. The effect of such exposure is then assessed. In particular, after such exposure, the sample is assessed to determine whether regulatory T cells (Treg) have been induced, in number and/or activity. The presence of Treg may be assessed in a number of ways as described herein. For example, the number of Tregs in a sample before and after exposure may be compared, or the amount of molecules that are characteristic of, or expressed by, Treg may be compared before and after exposure in order to determine whether any increase in Treg numbers or activity has been induced. Any one or more of these methods may be used in accordance with a method of the invention to assess the response of a sample of T cells to treatment with an anti-TNFα therapy.

In some embodiments, a method of the invention will involve detecting the presence of Treg. For example, where the sample of T cells from the patient contains no detectable Treg, no detectable Treg activity or no Treg of the type described herein, then the induction of such Treg upon treatment with the anti-TNFα therapy may be determined by simply detecting the presence or absence of such Treg or Treg activity. In other embodiments, the methods of the invention may involve the detection and also quantification of Treg or Treg activity. For example, the methods of the invention may detect a change in the number of Treg, a change in the number of particular types of Treg as described herein, a change in the characteristics of Treg in the sample, or a change in one or more activities of Treg. An increase in such numbers, characteristics or activity would be indicative that Treg have been induced as a result of the therapy.

In autoimmunity, CD4⁺CD25⁺Foxp3⁺ regulatory T cells are essential for the maintenance of tolerance. Depletion of Treg cells in mouse models is associated with the spontaneous development of a wide range of organ-specific diseases including inflammatory bowel disease and collagen induced arthritis (CIA). In humans, natural Treg, generated in the thymus, represent 3-5% of peripheral CD4⁺T cells, and express CD25, CTLA-4, CD62L and the transcription factor Foxp3.

The Tregs induced by a method of the invention may be CD62L⁻ and/or CCR7⁻, while expressing Foxp3, CD4, CD25 and CTLA-4. The presence of any of these markers, alone or in combination may be used to identify Tregs. For example, the presence of human Tregs may be determined by assessing the presence of Foxp3 expressing cells, cells expressing Foxp3 and CD4, T cells that do not express CD62L and/or CCR7, or cells that are Foxp3⁺, CD62L⁻ and CCR7⁻. The expression of CD127 in conjunction with CD25 and/or Foxp3 can be used to further define (and isolate) human Treg, since Foxp3⁺ Treg appear to localise within the CD25⁺CD127⁻ fraction of CD4⁺T cells. The presence of Tregs can therefore be identified by looking for CD25⁺CD127⁻ T cells in a sample, or by looking for CD25⁺CD127⁻ Foxp3⁺ cells or CD127⁻Foxp3⁺ cells.

A method of the invention may comprise detecting the presence of cells that are characterised by any one, two, three, four or more of the following markers: Foxp3⁺, CD4⁺, CD25⁺, CD127⁻, CD62L⁻, CCR7⁻, CTLA-4, GITR, TGFβ and LAP. For example, a method of the invention may involve the detection of cells that are: Foxp3⁺; Foxp3⁺ and CD4⁺; CD25⁺ and CD127⁻; and/or CD62L⁻ and CCR7⁻.

A method of the invention may involve the detection of cells that are Foxp3⁺CD127⁻. A method of the invention may further involve the identification of such cells that are also CD62L⁻ and CCR7⁻.

The presence of such T cell markers may be assessed by methods routine in the art. These may include expression- or function-based tests. For example, fluorescence activated cell sorting (FACS) can be used to isolate and identify cells that exhibit one or more such characteristics. FACS can thus be used to screen a sample for cells that exhibit a particular expression profile.

By exploring the mechanisms that underlie the increased number of Treg in rheumatoid arthritis patients following infliximab therapy, an insight may be found into how to harness regulatory T cells for the treatment of this disease. Similarly, such an analysis allows the identification of a variety of biomarkers that can be used to predict clinical response to therapy. One such example is Foxp3 expression, which may be measured using an overnight cell culture.

The mechanisms by which Treg mediate their effects are still not completely understood, and seem to vary according to the system under study, but in general natural Treg require direct cell contact and possibly the involvement of cytokines such as IL-10 and TGFβ.

It is established herein that the induction of Treg is dependent on TGFβ. This is in keeping with the actions of TGFβ, which can induce the differentiation of Treg from responder T cells. This process also relies on the presence of both IL-2 and CTLA-4. While not wishing to be bound by theory, it is hypothesised that the induction of Treg by infliximab is regulated by TGFβ production or responsiveness. Extending this hypothesis further, it can be predicted that the reason that there is no increase in the numbers of Treg in patients who have not responded to infliximab, is due to a failure of TGFβ production or function.

A method of the invention may therefore assess the presence of Treg by looking for a functional characteristic of Treg, such as any of the functions of Tregs discussed herein. For example, a method of the invention may assess the ability of T cells to suppress pre-inflammatory cytokine production by a population of responder T cells.

Cytokines that may be suppressed include TNFα, INFγ, IL-6 and IL-1. A method of the invention may assess the ability of T cells to suppress proliferation of a population of responder T cells.

A method of the invention may assess the presence of Treg by looking for molecules that are produced by or induced by Treg. For example, a method of the invention may involve assessing the presence of one or more pro-inflammatory cytokines that are characteristic of Treg, such as TGFβ or IL-10.

### Diagnostic Methods

The invention therefore provides diagnostic methods that allow an assessment of how, and whether, a patient will respond to treatment with an anti-TNFα therapy. As explained in more detail below, in some embodiments, this information may be used by a medical or veterinary practitioner in order to assist them in selecting a suitable course of treatment for the patient. In particular, this information may be useful in assisting them to decide whether or not to use the particular anti-TNFα therapy tested, or a similar therapy, on the patient in vivo.

The detection of induction in Treg activity or numbers as described herein indicates that the patient is likely to respond to treatment with that anti-TNFα therapy. This may also indicate that the same patient will respond to other anti-TNFα therapies. For example, induction in Treg activity or numbers in response to an anti-TNFα antibody therapy may indicate that the patient is likely to respond to treatment with that antibody, but may also indicate that the patient is likely to respond to treatment with other anti-TNFα antibodies, such as other antibodies that bind to TNFα. Thus, a patient that shows a positive response to infliximab in an in vitro test of the invention is likely to also respond to other therapies that act via a similar pathway to infliximab, such as other anti-TNFα antibodies.

A patient may be classified as likely to respond to therapy if he or she has a greater than 50% chance of responding to that therapy. Preferably the patient has a greater than 60%, greater than 70%, greater than 80%, greater than 90%, greater than 95% or more chance of responding to the given therapy. Preferably, a patient that is identified as likely to respond to therapy has an improved probability of responding to therapy than a patient that has not been tested. For example, infliximab is effective in 60-70% of patients with rheumatoid arthritis (RA). Preferably an RA patient that is identified by a method of the invention as being likely to respond to infliximab will have a greater than 60-70% chance of responding, such as greater than 70%, greater than 75%, greater than 80%, greater than 90%, greater than 95% or more.

The absence of an induction in Treg activity or number as described herein indicates that the patient is unlikely to respond to treatment with that anti-TNFα therapy. This may also indicate that the same patient will not respond to other anti-TNFα therapies. For example, the failure of an anti-TNFα antibody to induce Treg activity or number in accordance with the invention indicates that the patient is unlikely to respond to treatment with that antibody. It may also mean that the patient is unlikely to respond to treatment with other anti-TNFα antibodies. Thus, a patient that shows a negative response to infliximab in an in vitro test of the invention is also unlikely to respond to other therapies that act via a similar pathway to infliximab, such as other anti-TNFα antibodies.

A patient may be classified as unlikely to respond to therapy if he or she has a less than 50% chance of responding to that therapy. Preferably the patient has a less than 40%, less than 30%, less than 20%, less than 10%, less than 5% or less chance of responding to the given therapy. Preferably, a patient that is identified as unlikely to respond to therapy has an lesser probability of responding to therapy than a patient that has not been tested. For example, infliximab is ineffective in 30-40% of patients with rheumatoid arthritis (RA). Preferably an RA patient that is identified by a method of the invention as being unlikely to respond to infliximab will have a less than 30-40% chance of responding, such as less than 30%, less than 25%, less than 20%, less than 10%, less than 5% or less.

### Methods of Treatment

The present invention also relates to anti-TNFα therapies. Suitable therapies and conditions to be treated are discussed above. The invention relates in particular to methods for determining how a patient is likely to respond to a particular anti-TNFα therapy. These methods may be used as part of a therapeutic method. That is, a method of treatment of a patient with an anti-TNFα therapy may comprise the additional step of carrying out a predictive test as described herein in order to determine whether or not the patient is likely to respond to that therapy.

Accordingly, the invention provides methods for treating diseases or conditions associated with TNFα activity. For example, the invention provides a method for treating an inflammatory or autoimmune disease, the method comprising: (a) identifying a patient as being likely to respond to treatment with an anti-TNFα therapy using a predictive method as described herein; and (b) treating the patient with said anti-TNFα therapy. Suitable diseases, patients, and therapies for use in such a method are as described herein.

An anti-TNFα therapy as described herein, such as an anti-TNFα antibody, is also provided for use in the treatment of diseases or conditions associated with TNFα activity, such as an inflammatory or autoimmune disease, in a patient, wherein said patient has been identified as being likely to respond to treatment with an anti-TNFα therapy by a predictive method as described herein. For example, an anti-TNFα therapeutic agent as described herein, such as an anti-TNFα antibody, may be used in the manufacture of a medicament for the treatment of diseases or conditions associated with TNFα activity, such as an inflammatory or autoimmune disease, in such a patient. Again, suitable therapies, patients and diseases are as described herein.

The administration of the therapy may be for either “prophylactic” or “therapeutic” purpose. As used herein, the term “therapeutic” or “treatment” includes any of following: the prevention of disease or of symptom(s) associated with disease; a reduction or prevention of the development or progression of a disease or symptom(s); and the reduction or elimination of an existing disease or symptom(s).

Prophylaxis or therapy includes but is not limited to eliciting an effective anti-TNFα response and/or alleviating, reducing, curing or at least partially arresting symptoms and/or complications resulting from or associated with TNFα. When provided prophylactically, the therapy is typically provided in advance of any symptom. The prophylactic administration of the therapy is to prevent or ameliorate a subsequent symptom or disease. When provided therapeutically, the therapy is typically provided at or shortly after the onset of a symptom of disease. Such therapeutic administration is typically to prevent or ameliorate the progression of, or a symptom of the disease or to reduce the severity of such a symptom or disease.

Specific routes, dosages and methods of administration of anti-TNFα therapies may be routinely determined by the medical practitioner.

For example, infliximab is currently sold under the brand name Remicade®. Remicade® is sold for administration by intravenous infusion, typically at 2-month intervals and at a clinic or hospital. The recommended dose of Remicade® for treatment of rheumatoid arthritis is 3 mg/kg, followed by additional similar doses at 2 and 6 weeks after the first infusion and then every 8 weeks thereafter. For Crohn's disease, alkylosing spondylitis, psoriatic arthritis, psoriasis or ulcerative colitis, the recommended dose of Remicade® is 5 mg/kg over a similar administration schedule.

Adalimumab is currently sold under the brand name Humira®. Humira® is marketed in both preloaded 0.8 ml syringes and in preloaded pen devices, both for injection subcutaneously, typically by the patient at home. In accordance with the present invention, an anti-TNFα therapy may be administered via any suitable route in any suitable dose and in any suitable administration regime. The recommended dose of Humira® for adult patients with rheumatoid arthritis, psoriatic arthritis or ankylosing spondylitis is 40 mg administered every other week. The recommended Humira® dose regimen for adult patients with Crohn's disease is 160 mg initially at Week 0 (dose can be administered as four injections in one day or as two injections per day for two consecutive days), 80 mg at Week 2, followed by a maintenance dose of 40 mg every other week beginning at Week 4.

An anti-TNFα therapeutic agent may be employed alone as part of a composition, such as but not limited to a pharmaceutical composition or a vaccine composition or an immunotherapeutic composition to prevent and/or treat a condition associated with TNFα activity.

An anti-TNFα therapy as described herein may be used in combination with one or more other therapies intended to treat the same patient. By a combination is meant that the therapies may be administered simultaneously, in a combined or separate form, to a patient. The therapies may be administered separately or sequentially to a patient as part of the same therapeutic regimen. For example, an anti-TNFα therapy as described herein may be used in combination with another therapy intended to treat an inflammatory or autoimmune disease. The other therapy may be a general therapy aimed at treating or improving the condition of a patient with an inflammatory or autoimmune disease. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with an anti-TNFα therapy. For example, infliximab is commonly used in combination with methotrexate in the treatment of rheumatoid arthritis.

The other therapy may be a specific treatment directed at the particular disease or condition suffered by the patient, or directed at a particular symptom of such a disease or condition. For example, where the patient has rheumatoid arthritis, the treatment as described herein may comprise treatment with an anti-TNFα therapy, and also treatment with a further therapy specifically intended to treat, prevent or reduce the symptoms of the rheumatoid arthritis.

### Kits

The invention also relates to a combination of components described herein suitable for use in a predictive method of the invention which are packaged in the form of a kit in a container. Such kits may comprise a series of components to allow for a method of the invention. For example, a kit may comprise an anti-TNFα therapeutic agent, such as an anti-TNFα antibody, and means of detecting the presence or activity of regulatory T cells in a sample. The kit may optionally contain other suitable reagent(s), control(s) or instructions and the like to allow a method of the invention to be carried out. Such a kit may also comprise means for obtaining a sample from the patient.

### EXAMPLES

### Example 1

### In Vivo Methods

### Materials and Methods

### Study Population.

Twenty seven patients with active rheumatoid arthritis (RA), fulfilling the revised classification criteria of the American College of Rheumatology for RA were evaluated before and after anti-TNFα therapy (Infliximab given at a dose of 3 mg/kg i.v. at week 0, week 2, week 6, and then at every 8 weeks in combination with stable doses of methotrexate 7.5-15 mg/week orally) and stable non-steroidal anti-inflammatory drugs (NSAIDs). Patients taking prednisolone were excluded from the study since steroids are known to affect lymphocyte function and CD25 expression. Only patients with a disease activity score (DAS) of greater than 5.1 were treated with anti-TNFα therapy. Response was defined as a drop in the DAS of greater than 1.2. Patients who were responding to conventional dosages of methotrexate (15-25 mg/week) were also included in this study.

### Antibodies.

The following antibodies were used: FITC conjugated anti-CD4 (RPAT4), Cy-Chrome conjugated anti-CD25 (M-A251);

### Cell Isolation.

Human blood mononuclear cells (PBMC) were isolated by Ficoll-Paque Plus (Amersham Pharmacia Biotech, Piscataway, N.J.) gradient centrifugation. Cells were cultured in RPMI 1640 media supplemented with 2 nM L-glutamine, 5 mM HEPES, and 100 U/μg/ml penicillin/streptomycin, 0.5 mM sodium pyruvate, 0.05 mM nonessential amino acids (both from Life Technologies, Rockville, Md.), and 10% FCS (all from BioWhittaker, Walkersville, Md.) in 96-well U-bottom plates (Nunc, Corning). Non CD4⁺T cells were stained using a biotin antibody cocktail (10 μl/10⁷ total cells), incubated for 10 mins at 4° C. then magnetically labeled with anti-biotin micro-beads (20 μl/10⁷ total cells), incubated for 15 mins at 4° C., washed twice and depleted over a MACS LD column. CD4⁺CD25⁺ T cells were directly labeled with anti-CD25 micro-beads (10 μl/10⁷ CD4+ cells), incubated for 15 min at 4° C. and positively selected using MACS MS columns according to manufacturer's instructions (Miltenyi Biotec). T cell subpopulations of CD4⁺CD25⁺ T cells were used immediately after isolation. All the plate-bound anti-CD3 cultures received 1×10⁴ irradiated PBMC.

### Statistical Analysis.

All analysis for statistically significant differences was performed with Student's t test. P values <0.05 were considered significant.

### Results

### The Frequency of CD4⁺CD25^{high} T Cells is Higher in Anti-Tnfα Responding Patients Compared to the Same Patients Prior to Treatment.

The numbers of regulatory T cells were monitored in patients with RA, before and after treatment. The expression of CD25^{high/low on CD}4⁺ T cells was initially examined in four responding and four non-responding patients during the first three months of treatment using FACS analysis. Whereas the percentage of CD4⁺CD25^{high} T cells increased over time in those patients who responded to anti-TNFα therapy, there was no change in the percentage of CD4⁺CD25^{high} T cells in the four non-responding patients. The percentage of this population continued to rise in three out of the four responding patients six weeks after the first infusion of Infliximab and remained stable after three months of treatment. The subsequent analysis in a larger cohort of patients, including those responding to methotrexate therapy, revealed that the percentage of both CD4⁺CD25^{high or low} T cells, was significantly increased in anti-TNFα responding patients compared to the levels measured in those with active RA, methotrexate treated patients, and healthy controls. Active RA patients also showed an increased percentage of CD4⁺CD25^{low} T cells.

The methotrexate treated individuals were matched to the responding anti-TNFα treated patients with respect to their response to treatment. No reduction in numbers of regulatory T cells was measured in patients with active RA compared to healthy individuals. No differences in CD4⁺CD25^{high/low} expression were detected in patients with RA who failed to respond to anti-TNFα therapy compared to healthy controls. A significant correlation between the CRP, a measure of rheumatoid disease activity, and the percentage of CD4⁺CD25^{high} T cells (but not CD4⁺CD25^{low}) confirmed the relationship between a response to anti-TNFα therapy and an expansion in this cell population. There was no significant change in the absolute numbers of CD4⁺T cells in PBMC before treatment and 3 months after anti-TNFα treatment.

### Example 2

### Treg in RA Patients

### Materials and Methods

### Patient Population.

31 patients with active RA, fulfilling the revised classification criteria of the American College of Rheumatology for RA were evaluated before and 4-6 months after anti-TNFα therapy. 20 Healthy individuals were used as controls. This study was approved by the UCLH Ethics Committee.

### Antibodies.

The following Abs were used: FITC-anti-CD4 (RPA-T4), PE-Cy5-anti-CD25 (M-A251), PE-anti-CD62L (Dreg-56), PE-Cy7-anti-TNF-α (Mab11), PE-Cy7-anti-IFN-γ (45.B3), PE-FoxP3 (PCH-101), APC-anti-CCR7 (3D12), PE-anti-CD45RO (UCLH1). T cells were activated with soluble anti-CD3 (HIT-3a) and anti-CD28 (CD28.2) as indicated. All antibodies were from BD Bioscience (except FoxP3, from eBiosciences). For neutralisation experiments, anti-TGF-β1 (9016.2) and anti-human IL-10 (25209) were used (R&D systems). Infliximab, a chimeric IgG1 anti-TNFα monoclonal antibody, was donated by Schering Plough.

### Cell Isolation.

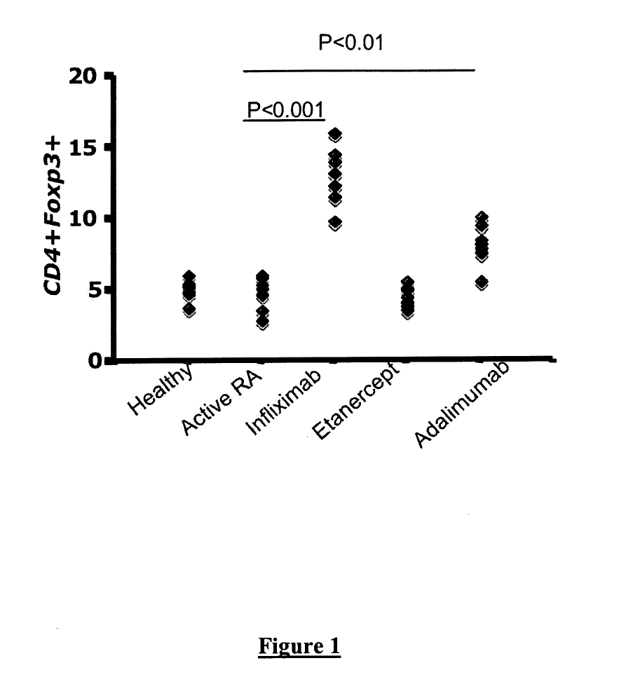

Human blood mononuclear cells (PBMC) were isolated by Ficoll-Paque (GE Healthcare) and cultured in RPMI-1640 with 100 U/μg/ml penicillin/streptomycin (Life Technologies) and 10% FCS (Sera Laboratories International, Ltd). Magnetic-bead separation was carried out with all magnetic columns and magnetically-labelled beads purchased from Miltenyi Biotec GmbH. In order to purify cells using FACS sorting (Moflo), cells were stained with the above conjugated antibodies (CD4, CD25 and CD62L). Cells were sorted according gates as indicated in Supplementary FIG. 1.

### Flow Cytometric Analysis and Cytokine Detection.

Cells were surface stained as previously described (11), using the above mentioned conjugated antibodies (CD4, CD25 and CD62L). For intracellular analysis of TNF-α and IFN-γ production, cells were cultured at 2×10⁵ for 48 h, with PMA, ionomycin and Golgi Plug added in the final 5 h of culture. TGF-μ1 was quantified using an ELISA kit (R&D Systems). In some experiments 24-well Transwell plates (Costar) with 0.4-μm membrane supports were used.

### Proliferation Assays.

In all experiments, regulatory T cells were cultured at 2×10⁵ cells/ml, with CD4⁺CD25⁻ T cell numbers adjusted accordingly for ratio experiments. Cells were cultured in 96-well U-bottomed plates (Nunc) for 5 days with ³H-thymdine added in last 18 h of culture. Proliferation was measured using a liquid scintillation counter.

### Statistical Analysis.

Statistical significance was determined using Student's T-test, with p values <0.05 regarded as statistically significant.

### Results and Discussion

The Expanded Population of Treg from Infliximab Treated Patients is Foxp3⁺ and CD62L⁻

Peripheral CD4⁺T cells isolated from healthy, active RA and post infliximab patients were analyzed for the expression of the transcription factor Foxp3 by intracellular staining. A 2-3 fold increase in the percentage of CD4⁺Foxp3⁺ cells was observed in the PBMC of post-infliximab patients, compared to active RA patients or healthy individuals, suggesting that the increase in CD25 expression in post-anti-TNFα treated patients does reflect increased numbers of Treg. There was no significant difference in FoxP3 expression in CD4⁺T cells from active RA patients compared to healthy controls. To better characterize the phenotype of the Treg after infliximab, we measured surface markers representative of activation, memory and regulation. A significant increase in the percentage of CD4⁺CD25^{hi}CD62L⁻ was observed in PMBC from infliximab patients, compared to RA patients with active disease prior to infliximab and healthy individuals.

We next assayed the expression of CD62L in the CD4⁺Foxp3+ population. Whereas most CD4⁺Foxp3⁺ cells from healthy individuals and patients with active RA expressed CD62L, the profile of expression was remarkably different following infliximab treatment, where the majority of CD4⁺Foxp3⁺ cells expressed low levels of CD62L. There was no change in percentage of Foxp3⁺ cells or shift in CD62L expression in patients responding to methotrexate. In comparison to the Tregs found in healthy individuals and patients with active disease, RA Tregs post-infliximab expressed CD45RO, but had a reduced expression of CCR7. Further examination of the CD4⁺Foxp3⁺CD62L⁻RA Tregs post-infliximab revealed that these cells lacked CCR7 expression and remained CD45RO⁺.

CD62L⁻ Treg from Infliximab Treated Ra Patients Mediate their Suppressive Action Through TGFβ and Il-10.

CD4⁺CD25^{hi} were FACS sorted according to their expression of CD62L (identified as CD62L⁺ and CD62L⁻ Treg, and their regulatory capacity was assessed with respect to inhibition of proliferation, TNFα and IFNγ production by autologous CD4⁺CD25⁻ T cells. CD62L⁺Treg isolated from healthy individuals were more potent at suppressing CD4⁺CD25⁻ T cell proliferation than CD62L⁻Treg. Conversely, CD62L⁻Treg isolated from RA patients post infliximab, exhibited a more potent suppression of T cell proliferation than their CD62L⁺Treg counterparts. Similarly, we observed a “switch” in the suppressor population from CD62L⁺Treg in healthy individuals to CD62L⁻Treg in patients post infliximab, with respect to inhibition of IFNγ and TNFα production. There was a significant reduction in the suppressor potency of CD62L⁺Tregs isolated from patients with active RA compared to healthy individuals, confirming that CD4⁺CD25^{hi} Tregs are defective in RA patients. Of importance, the potency of the CD62L⁺Tregs after anti-TNFα therapy was not restored to levels found in healthy controls.

Treg were co-cultured with autologous CD4⁺CD25⁻ T cells in the presence of neutralizing mAbs to TGFβ and IL-10, previously recognized as regulatory cytokines involved in the suppressive mechanism of adaptive Treg. In healthy individuals the suppressive effect of CD62L⁻Treg was unaltered by the neutralization of TGFβ or IL-10. In contrast, neutralization of TGFβ, and to some extent IL-10, significantly impaired the suppressive capacity of the CD62L⁻Treg from post infliximab treated patients. When the action of both TGFβ and IL-10 were blocked, the suppressive activity of these CD62L⁻Treg was almost abolished. The potency of CD62L⁻ Treg from healthy individuals was unaltered by blockade of IL-10 or TGFβ.

We assayed the functional properties of purified CD62L″ Treg, isolated from RA patients post-infliximab therapy, to dissect out their mode of action. Cell contact was required to effect maximal suppression of TNFα and IFNγ production by CD4⁺CD25⁻T cells, though some cytokine suppression is still observed when cell contact is prevented. The fact that the Treg isolated from patients post-infliximab required both cell contact and cytokines for their suppressive effect could be explained by data indicating that TGFβ can mediate suppression through cell contact, when the predominant form of TGFβ is membrane bound. In addition, in some experimental systems IL-10 production also depends on cell contact between Treg and CD4⁻CD25⁻ T cells. Collectively these results suggest that infliximab therapy gives rise to, or recruits from the periphery, a population of Treg that phenotypically and functionally differ from natural Treg, present in healthy individuals or in patients with active RA.

In Vitro Infliximab Stimulation of CD4⁺CD25⁻ T Cells from Ra Patients Induced a CD62L⁻ Treg population.

Infliximab was added in vitro to CD4⁺CD25⁻ T cells isolated from active RA or healthy individuals, and the expression of Foxp3 measured by intracellular staining. The addition of infliximab to purified active RA CD4⁺CD25⁻ T cells resulted in a substantial increase in the percentage of CD4⁺Foxp3⁺ cells, whereas no such effect was seen when CD4⁺CD25⁻ T cells were isolated from healthy individuals. Neutralization of TGFβ in this culture system completely prevented the differentiation of CD4⁺Foxp3⁺T cells population from CD4⁺CD25⁻ T cells. Moreover, infliximab induced a significant increase in TGFβ production from CD4⁺CD25⁻ T cells from patients with active RA, but not healthy individuals. To determine whether infliximab modulates natural Treg Foxp3 expression, CD4⁺CD25^{hi} T cells were isolated from healthy individuals or patients with active RA and cultured with infliximab. The results showed that infliximab did not affect the expression of FoxP3 on CD4⁺CD25^{hi} Treg, supporting the evidence that this agent targets CD4⁺CD25⁻ T cells, rather than modulating pre-existing Tregs. We next measured CD62L expression following culture of CD4⁺CD25⁻ T cells with infliximab. The addition of infliximab to CD4⁺CD25⁻ T cells from active RA patients, but not from healthy individuals, led to a reduction in CD62L expression. Thus, these in vitro infliximab generated T cells share reduced CD62L expression as a common feature with the Tregs present in PBMC of RA patients that had received infliximab therapy.

We sought to confirm that the Foxp3+ T cells differentiated upon infliximab stimulation possess suppressor activity. Since the majority of purified CD4⁺CD25⁻ T cells had acquired CD25 expression when stimulated with anti-CD3/CD28, with or without infliximab, the CD4⁺CD25⁺T cells were purified, and cultured with freshly isolated autologous CD4⁺CD25⁻ T cells. Only the CD4⁺CD25⁺T cells derived from active RA patients, but not healthy individuals, which had been co-cultured with infliximab, were able to suppress production of IFNγ and TNFα from freshly isolated CD4⁺CD25⁻ T cells.

These data suggest that the naturally derived Tregs, defined as CD4⁺CD25^{hi}CD62L⁺, appear to remain defective following treatment with anti-TNFα therapy. The restoration of function of the CD4⁺CD25^{hi} population following infliximab therapy is most likely to be due to the suppressive effects of the newly differentiated CD62L⁻ Tregs.

### Example 3

### In Vitro Predictive Methods

### Patient Selection

50 patients with rheumatoid arthritis (RA), who are to begin anti-TNFα therapy (including infliximab and etanercept) in the Bloomsbury Rheumatology Unit's New Therapies Clinic are recruited to the study. RA patients to be studied fulfil British Society of Rheumatology (BSR) eligibility criteria for the introduction of biologic therapies, with a Disease Activity Score (DAS) of >5.1, seropositive for anti-cyclic citrullinated peptide (CCP) and/or rheumatoid factor (RF). Response to therapy in RA patients is defined using criteria established by the European League Against Rheumatism measured measured twice at 3-5 months (DAS is also used as a continuous variable to assess response). The change in CRP is also documented. Blood samples are taken before therapy, and then 2, 8 weeks and 4 months following therapy (blood taken on no more than 4 occasions from individual patients).

A group of patients with psoriatic arthritis (peripheral joint disease only) is also studied. Their disease activity and response to therapy is assessed as recommended by the BSR (Kyle et al (2005) Rheumatology 44: 390-397).

Controls include 30 patients with RA, both pre- and post-methotrexate (MTX) treatment, as well as 30 healthy volunteers. Patients are on stable doses of non-steroidal anti-inflammatory drugs. Patients taking prednisolone are excluded from the study since steroids are known to affect lymphocyte function and CD25 expression. Patients and healthy control are age and sex matched.

Venous blood (50 to 80 ml) is collected, into EDTA—as anti-coagulant, from patients prior to commencing therapy. Patients subsequently have blood taken on attending the clinic either for an infusion or for routine follow-up. Patients do not have blood taken for the study on more than 4 occasions. We have previously determined that one freeze/thaw cycle of cells does not compromise regulatory or responder T cell function. Consent is obtained from all individuals. Ethical approval has been obtained from the UCLH ethics committee. There are two specialist nurses attached to the New Therapies Clinic who assesses the patients' response to therapy and takes blood samples thus greatly facilitating the project.

1. Establishing Whether the Induction of Regulatory T Cells by Infliximab or Adalimumab In Vitro is Associated with Clinical Response, and More Importantly, can Predict Response in RA Patients.

Responder CD4⁺ T cells are isolated by FACS sorting (CD4⁺CD25⁻CD127⁺) from RA patients with active disease before commencement on infliximab or adalimumab therapy. The responder T cells are activated overnight in vitro (2 μg/ml anti-CD3/CD28) with or without 10 μg/ml infliximab or adalimumab, and Foxp3 induction is assayed by FACS (eBioscience). The degree of in vitro induction of Treg is correlated with the clinical response of the same patients to infliximab therapy at 3 months. Response is also confirmed 8 weeks later prior to the next infusion. An increase in Treg numbers in this in vitro assay should predict clinical response to therapy.

The number of CD4⁺Foxp3⁺ cells in vivo in PBMC is assessed after infliximab therapy to determine the correlation between in vivo and in vitro induction of Tregs.

PBMC were isolated from patients with RA before and after (6 weeks, 3 and 6 months) therapy with infliximab or adalimumab. The numbers of CD4⁺Foxp3⁺ cells were measured by FACS staining. The numbers of Foxp3⁺CD4⁺ T cells in patients who were responding to treatment were compared with those detected in the pre-treatment, healthy, and non-responding patients (see FIG. 1). The results in FIG. 1 show the numbers of regulatory T cells in the peripheral blood (identified as CD4⁺Foxp3⁺) of patients with rheumatoid arthritis following 3 months treatment of infliximab or adalimumab. Both infliximab and adalimumab led to an increase in the number of regulatory T cells in patients responding to therapy. Only those patients who responded to therapy are shown in FIG. 1. This shows that the stimulation of regulatory T cells in the blood of RA patients treated with infliximab or adalimumab is predictive of whether or not those patients will respond to the therapy.

We have shown that the induction of Foxp3 in the responder T cell population correlates with increase in the number of regulatory cells as assessed by functional assays. As an alternative to Foxp3, CD25 and CD127 are also used to enumerate Treg numbers. The regulatory nature of the Foxp3⁺CD4⁺T cells is also assessed by purifying the resulting CD4⁺CD25⁺CD127 T cells that arise in the in vitro cultures, and testing their ability to suppress TNFα and IFNγ production (assayed by intracellular cytokine production) and proliferation (assayed by CFSE) by freshly isolated responder (CD4⁺CD25⁻CD127⁺) T cells. Assumptions made to estimate the number required for this study using 90% power, and a significance level of 5% are that 90% of those with positive test (increase of Treg by >5%) will be responders and 30% with a negative test will be responders to treatment.

2. Determining Whether the Induction of Tregs in RA Patients Also Occurs with Etanercept In Vivo and In Vitro.

The Treg function in RA patients is assessed before and after treatment to determine whether the ability of Treg to suppress T cell function changes following etanercept treatment. Specifically, the experiment addresses whether the defect in Treg function in RA patients, namely the inability to suppress pro-inflammatory cytokine production is restored following etanercept therapy.

PBMC were isolated from patients with RA before and after (6 weeks, 3 and 6 months) therapy with etanercept. The numbers of CD4⁺Foxp3⁺ cells were measured by FACS staining. The number of Foxp3⁺CD4⁺ T cells in patients who were responding to treatment, were compared with those detected in the pre-treatment, healthy, and non responding patients.

The numbers of regulatory T cells in the peripheral blood (identified as CD4⁺Foxp3⁺) of patients with rheumatoid arthritis following 3 months treatment with etanercept were analysed. FIG. 1 shows the percentage of such regulatory T cells in PBMC from these samples. Only responding patients are shown for the etanercept treated group. FIG. 1 shows that etanercept treatment did not lead to an alteration in regulatory T cell numbers despite patients responding clinically to the same degree as treatment with infliximab or adalimumab. This implies that etanercept may have a different mechanism of action compared to infliximab and adalimumab. This also implies that assessment of regulatory T cell numbers or activity may not be a suitable marker for the likely responsiveness of a patient to treatment with etanercept.

The effects of etanercept on Treg numbers from patients with psoriatic arthritis is also assessed.

We have shown that Tregs induced by infliximab are phenotypically distinct in that they have reduced expression of CD62L and CCR7 compared to natural Treg. Tregs are further characterised in RA patients before and after etanercept using a number of relevant markers including CTLA-4, GITR, CD62L, CCR7, CD25, CD127.

The ability of Treg isolated from RA patients to suppress pro-inflammatory cytokine production and proliferation by responder T cells before and after etanercept therapy is assessed. The various cell populations are sorted by FACS (Aria, BD). In order to maximise the quantity and purity of Tregs by FACS sorting, antibodies directed against CD25 and CD127 are used. Thus, CD4⁺CD25⁺CD127⁻ Treg, or CD4⁺CD25⁻CD127⁺ responder T cells, or a mixture (1:1; 1:3 co-cultures) are stimulated with anti-CD 3/CD28 and monitored for proliferation by thymidine incorporation and CFSE. Cytokine production (TNFα, IFNγ, TGFβ, IL-10) will be measured by intracellular cytokine production and/or ELISA/CBA in these Treg suppression assays.

This experiment examines whether the inhibitory effect of Tregs present after etanercept therapy relies on cell contact (using a transwell membrane) or through the effect of inhibitory cytokines such as TGFβ or IL-10 using blocking antibodies. Since recent attention has focussed on IL-17 as an important pro-inflammatory cytokine production of this cytokine by responder T cells from RA patients is also measured as well as in the inhibition assays where Tregs are present. Where appropriate, the results of these experiments are compared to those obtained with healthy individuals as well as RA patients treated with infliximab.

These experiments also investigate whether etanercept can induce the generation of Treg from responder T cells in vitro. Responder (CD4⁺CD25⁻CD127⁺) T cells are purified by FACS sorting from patients with RA and healthy controls and the ability of etanercept to induce Treg in vitro is assessed as described above for infliximab.

Various concentrations of etanercept are used as well as differing activation conditions (±IL-2, anti-CD3). Where Foxp3⁺ Treg are induced they are purified and their phenotype and suppressive potency in suppression assay with autologous responder T cells assessed. These results are compared with information gleaned from studying Treg after etanercept therapy in vivo. The next step involves determining whether TGF is important for this effect (as has been shown for infliximab) by using blocking antibodies as well as assaying for the production of TGFβ by ELISA and surface staining. The effect of etanercept on natural Tregs (Foxp3 expression) after their isolation by FACS sorting is also assessed.

These experiments shed light on the differing mechanisms of action of infliximab compared to etanercept providing an explanation for their differential efficacy and a rationale for their differing therapeutic use. The induction of Treg by infliximab therapy may uniquely lead to restoration of tolerance in RA. Thus we would predict that whilst infliximab can lead to disease remission if given to patients with early RA, etanercept would not have such an effect.

3. Determining Whether Patients with Active Psoriatic Arthritis have Defective Treg and if Peripheral Blood Treg Numbers Increase Following Infliximab Therapy.

Treg function is measured in patients with psoriatic arthritis to determine any functional impairment using standard in vitro assays as described above. It has been reported that Tregs from patients with psoriasis are defective and it is likely that Tregs from patients with psoriatic arthritis also display this abnormality. The induction of Tregs by infliximab therapy is tested by enumerating the number of CD4⁺Foxp3⁺ T cells after 3 months treatment. In parallel the induction of Foxp3 expression by infliximab in responder T cells in vitro is also assessed, as has been established for responder T cells derived from RA patients, as well as the suppressor activity of any such induced Treg.

4. Exploring the Mechanisms that Underlie the Induction of Treg by Infliximab.

The next set of experiments is guided by the results obtained in the previous sections. Preliminary data indicate that Treg induction is specific to RA patients who respond to infliximab therapy. We propose that the key discriminatory factor that governs Treg induction by infliximab is TGFβ production. This may act as a surrogate indicator of Foxp3 induction, representing a simper test to predict clinical response to infliximab.

We already have shown that the induction of Treg by infliximab is dependent on TGFβ, a regulatory cytokine known to be involved in the induction of Treg. This experiment determines the expression of TGFβ latency associated peptide (LAP) on responder, CD4⁺CD25⁻CD127⁺, T cells (ex vivo or activated overnight with anti-CD 3/CD28) from healthy controls, RA patients and patients with psoriatic arthritis by FACS. By comparing the ability of infliximab to induce Treg from responder T cells derived from different patient groups, a correlation between TGFβ (LAP) expression on responder T cells and induction of Treg is correlated. TGFβ production is also measured by ELISA. As an alternative hypothesis, it is possible that differential responsiveness to TGFβ rather than its expression, governs the induction of Foxp3. Therefore to gain insight into the consequences of TGFβ signalling on the responder T cells in the different patient groups, TGFβR expression is assayed by FACS and the activation of the Smad signalling cascade by Western blotting. The latter focuses on Smad2 and 3 phosphorylation. TNFα is also known to interfere with TGFβ mediated signalling, via effects on TGFβRII expression and downstream signalling. The induction of these signalling proteins in responder T cells alone (from the different patient groups) is assessed with or without addition of infliximab, using TGFβ blocking antibodies to target this pathway. In addition, TGFβ is added to responder T cells to establish the potency of this cytokine to induce Foxp3 expression in responder T cells from the different patient groups.

CTLA-4 and IL-2 are both required for the TGFβ dependent induction of Treg from responder (CD4+CD25−CD127+) T cells (30-32). Therefore CTLA-4 expression by FACS and IL-2 production by FACS (intracellular staining) and/or ELISA during responder T cell activation from the different patient groups (RA responder, non-responder, psoriatic arthritis responder) is assessed, with or without infliximab present in the culture. These findings are correlated with CTLA-4 expression in Tregs from RA patients before and after infliximab therapy. Since TGFβ has been shown to upregulate CD80 expression on responder T cells (32), CD80 expression on activated responder T cells is also assessed in this experiment.

Having established the connection between infliximab induction of Treg and TGFβ, we will next address the plasticity of the TGFβ response in the generation of Treg as opposed to pro-inflammatory Th17 cells in the context of RA. A number of factors appear to regulate Th17 differentiation including IL-2, TGFβ, and IL-6 (33-35). We hypothesise that infliximab, by downregulating both TNFα and IL-6, drives the inflammatory Th17 response towards a regulatory phenotype. We have preliminary evidence that IL-17 is produced in co-cultures of Treg and responder T cells from patients with RA. These co-cultures were stimulated with anti-CD3/CD28 but in the absence of monocytes. In order to obtain a complete picture of the conditions required for the generation of IL-17 in the co-cultures, the production of TGFβ, IL-6, IL-2, TNFα and IL-17 by responder T cells and Treg (separately or responder T cells/Treg combined) isolated from patients with active RA is compared to healthy individuals and RA patients treated with infliximab. The production of cytokines is measured by ELISA and/or intracellular staining. The individual contribution of IL-6, TNFα, TGFβ, and IL-2 to IL-17 production is assessed using the appropriate blocking antibodies. Suppression of T cell proliferation and TNFα/IFNγ production are also determined. The number of CD4⁺Foxp3⁺ T cells in these cultures is measured. Consequently, where responder T cells are incubated alone, these experiments mimic the in vitro expansion of Treg by infliximab and test whether, for example, IL-6 inhibition also induces the generation of Foxp3⁺CD62L⁻ T reg in a similar manner to infliximab. It is clear, at least in mice, that co-culture of Treg and responder T cells in an inflammatory environment promotes the formation of Th17 cells. Thus this experiment tests the capacity of Treg from patients with active RA, as opposed to healthy individuals, to support the production of IL-17 in co-culture experiments. These results are compared with the effects of Treg from post-infliximab patients, which suppress Th1 responses via TGFβ and IL-10, on Th17 production. The combination of IL-10 and TGFβ together could dampen Th17 differentiation, or, as recently revealed, these effects could be modulated by IL-2. Indeed, infliximab is known to increase IL-2 production by stimulated responder T cells.

5. Establishing Whether Regulatory T Cell Numbers Change in Secondary Lymphoid Organs after Anti-TNFα Treatment.

We have shown that, analogous to our data in RA, CD4⁺CD25⁺ Treg numbers increase in mice with collagen induced arthritis (CIA) after treatment with anti-TNFα. DBA/1 mice, immunized with type II collagen (CII) in Complete Freund's Adjuvant (CFA) as previously described (Feldmann et al (1996) Ann Rev Immunol 14: 397-440), are used in order to establish whether the increased number of Treg is also observed in the secondary lymphoid organs.

On the day of onset of arthritis mice are treated with anti-TNFα (goat anti-mouse TNF neutralizing antibodies i.p., R&D Systems; or monoclonal anti-TNF clone TNF-19.12, eBiosciences), isotype matched control or PBS control twice weekly for two weeks following the originally described protocol (Williams et al (1992) Proc Natl Acad Sci USA 89: 9784-9788). The level of CD4⁺CD25⁺Foxp3+ Treg in PBMCs is measured at regular intervals, using FACS staining, starting from the day of disease onset until disease resolution (0, 7, 14 and 21 days post treatment). At the end of the experiment (21 days after disease onset), the thymus, spleen and lymph nodes are excised from the mice and the levels of CD4⁺CD25⁺Foxp3⁺ T cells is measured.

Increased Treg after anti-TNFα are characterised by looking at the expression of surface markers (CTLA-4, CD62L, GITR etc.) and at their immunoregulatory (TGFβ, IL-10) cytokine profile. The comparison of the absolute number of Treg determined in the various compartments (i.e joints versus LN or spleens) together with the analysis of their phenotype (naïve versus memory) indicates the origin of the induced T reg (i.e. recruited from the site of inflammation as opposed to generated de novo). This experiment also assesses whether significant changes in Treg numbers are associated with any stage of disease (early arthritis, acute or remission phase). The response of Treg cells to CII±IL-2, and to anti-CD3/CD28 stimulation is also assessed, as well as testing their regulatory capacity through their ability to suppress CD4⁺CD25⁻ T cell proliferation and pro-inflammatory cytokine production.

6. Evaluation of the In Vivo Contribution of CD4⁺CD25⁺ Regulatory T Cells to the Resolution of Inflammatory Arthritis in DBA/1 Mice Treated with Anti-TNFα.

To test the hypothesis that anti-TNFα therapy may work, in part, through induction of Treg, DBA/1 mice are deplated of CD4⁺CD25⁺T cells and their response to anti-TNFα therapy compared with that of with non-depleted DBA/1 mice.

Mice are depleted of CD25-expressing cells (before commencement of anti-TNFα therapy) using an intraperitoneal injection of a depleting anti-CD25 monoclonal antibody (mAb) from the cell line PC61 as previously described Morgan et al (2003) Arthritis Rheum. 48: 1452-1460). Complete depletion of CD25-expressing T cells normally occurs 3 days after a single injection of anti-CD25, after 3 weeks 50% of the cells are recovered with full recovery typically after 4 weeks.

This regime may eliminate endogenous Treg, whilst sparing “induced” Treg, which could be generated from responder T cells in response to anti-TNFα treatment. Indeed our human studies suggest that infliximab induces Treg differentiation from responder T cells. To address whether induced Treg, rather than endogenous, are central to the protective effects of anti-TNFα, an additional two dose of anti-CD25 depleting antibodies are administered around 5 and 10 days after treatment with anti-TNFα. On the day of disease onset both depleted and non-depleted mice are treated with anti-TNFα, isotype matched control or PBS control. The severity of disease in mice depleted of CD4⁺CD25⁺ T cells is compared with the severity of disease in non-depleted mice both treated with anti-TNFα. Severity of disease will be assessed as previously described (Mauri et al (1996) Eur J Immunol 26: 1511-1518).

It can be difficult to distinguish between the importance of endogenous T reg and induced T reg due to the long half-life of anti-CD25 mAb (2-3 weeks). To circumvent this problem a transfer experiment is carried out, using the SCID model for arthritis which we and others have previously validated (Williams et al (1992) Clin Exp Immunol 88: 455-460; Mauri et al (2000) Nat Med 6: 673-679).

SCID mice are firstly reconstituted with arthritogenic splenocytes, or with arthritogenic splenocytes depleted of endogenous Treg (Miltenyi kit). The day after reconstitution, mice are treated with anti-TNFα. The severity of arthritis, together with the generation of Treg (CD4⁺CD25⁺Foxp3⁺), is evaluated and compared to control mice. Where Treg arises, determined by the increase of CD4+CD25+Foxp3⁺ T cells, the distribution of Treg in the blood, LN, spleens, and joints is then assessed.

To confirm their suppressive capacity, CD4⁺T cells (isolated from SCID mice differentially treated) are co-cultured with freshly isolated wild type arthritogenic CD4⁺CD25⁻ cells (T responder) in the presence of CII or anti-CD3/CD28. To further dissect whether anti-TNFα works via endogenous or induced T reg, SCID mice are also treated with anti-CD25 depleting antibodies 3-5 days after commencement of anti-TNFα administration to eliminate eventual “new” Treg.

1. A method for predicting the response of a patient to treatment with an anti-TNFα antibody, the method comprising
(a) providing an in vitro sample of T cells from the patient comprising CD4⁺CD25⁻ T cells;
(b) exposing said T cells to an anti-TNFα antibody; and
(c) determining whether regulatory T cells are induced in said sample of T cells wherein the induction of regulatory T cells indicates that the patient is likely to respond to treatment with said anti-TNFα antibody.

2. A method according to claim 1 wherein said patient has an inflammatory or autoimmune disease.

3. A method according to claim 2 wherein said patient has rheumatoid arthritis, psoriatic arthritis, Crohn's disease, psoriasis, ankylosing spondylitis or ulcerative colitis.

4. A method according to claim 1 wherein said anti-TNFα antibody is an anti-TNFα monoclonal antibody.

5. A method according to claim 4 wherein said anti-TNFα antibody is infliximab or adalimumab.

6. A method according to claim 1 wherein said sample of T cells comprises CD4⁺CD25⁻CD127⁺ T cells.

7. A method according to claim 1 wherein said sample of T cells is obtained by FACS sorting of a sample from the patient.

8. A method according to claim 1 wherein step (c) comprises assessing for the expression of one or more markers selected from Foxp3, CD4, CTLA-4, GITR, CD62L, CCR7, CD25, TGFβ, LAP and CD127 by said T cells.

9. A method according to claim 8 wherein step (c) comprises one or more of the following:
(a) assessing for the presence of Foxp3⁺T cells in said sample;
(b) assessing for the presence of Foxp3⁺CD4⁺ T cells in said sample;
(c) assessing for the presence of Foxp3⁺CD127⁻ T cells in said sample;
(d) assessing for the presence of CD25⁺CD127⁻T cells in said sample; and
(e) assessing the presence of CD62L⁻ and CCR7⁻ T cells in said sample.

10. A method according to claim 1 wherein the presence of regulatory T cells is determined by FACS.

11. A method according to claim 1 wherein step (c) comprises one or more of the following:
(a) assessing the ability of T cells in said sample to suppress pro-inflammatory cytokine production by a population of responder T cells; and
(b) assessing the ability of T cells in said sample to suppress proliferation of population of responder T cells.

12. A method according to claim 11 wherein said pro-inflammatory cytokine is selected from TNFα, INFγ, IL-6 and IL-1.

13. A method for treating an inflammatory or autoimmune disease, the method comprising:
(a) identifying a patient as being likely to respond to treatment with an anti-TNFα antibody by a method according to claim 1; and
(b) treating the patient with said anti-TNFα antibody.

14. (canceled)
